(19) United States
(12) Patent Application Publication
(10) Pub. No.: US 2021/0375425 A1
(43) Pub. Date: Dec. 2, 2021

(54) MULTIFUNCTIONAL INTELLIGENT FITNESS AND PHYSIOTHERAPY DEVICE

(71) Applicant: Macvon LLC, Moorestown, NJ (US)

(72) Inventor: Xing Zhang, Moorestown, NJ (US)

(21) Appl. No.: 16/998,984

(22) Filed: Aug. 20, 2020

(30) Foreign Application Priority Data
May 28, 2020 (CN) 202010464661.5

(51) Int. Cl. G16H 20/30; A63B 21/005; A63B 21/00; A63B 24/00; A63B 71/06; A63B 21/16; A63B 23/035; A63B 23/12; H04N 5/247; G06K 9/00

(52) U.S. Cl. CPC G16H 20/30; A63B 2220/05; A63B 21/153; A63B 24/0062; A63B 24/0087; A63B 71/0622; A63B 21/169; A63B 21/4035; A63B 23/03541; A63B 23/12; A63B 24/0075; H04N 5/247; G06K 9/00369; A63B 2071/065; A63B 2071/0647; A63B 2230/06; A63B 2225/50; A63B 2220/833; A63B 2071/0675; A63B 2220/806; A63B 21/0058

(57) ABSTRACT

A multifunctional fitness device comprising a fitness device body, a support arm and an intelligent control system, with a display device being disposed on a front surface of the fitness device body, the display device being a mirror display screen having functions of video teaching and ordinary mirror, a camera device being disposed at the top of the display device, and the camera device including a micro-camera and an infrared camera, support arms being disposed at both sides of the fitness device body through sliding rails respectively, a gear groove being disposed on the support arm, a gear fixing device for use in cooperation with the gear groove being disposed on the sliding rail, a rope being disposed inside the support arm, the other end of the rope being connected with an intelligent motor, which motor produces resistance to provide a resistant force to the support arm.

### BACKGROUND OF THE INVENTION

### 1. Field of the Invention

The present invention relates to the field of fitness appliance technologies and in particular to a multifunctional intelligent fitness and physiotherapy device.

### 2. Description of the Related Art

Along with continuous improvement of life, people have increasingly broad demands for fitness and physiotherapy. At present, the fitness and physiotherapy appliances or devices are mostly non-intelligent products. Further, the existing fitness and physiotherapy devices are of frame structure with counterweight as metal block. Due to the frame structure, the fitness device occupies a larger area and is inconvenient to install. With the traditional metal block counterweight, the fitness and physiotherapy device cannot achieve stepless adjustment to the counterweight, resulting in inconvenience of use. Also, for some household fitness appliances, the users cannot determine whether their exercises conform to requirements. Further, there is no device available on the market to provide group practice virtually on a big screen.

### SUMMARY OF THE INVENTION

Therefore, one object of the present application is to provide a multifunctional fitness and physiotherapy intelligent device which occupies a smaller area and can achieve intelligent control and provide intelligent feedback.

According to the present application, a fitness device comprises:
a fitness main body;
at least one rope slidably attached to the fitness main body, wherein the rope has two ends, with one end being connected to a handle, and the other end being connected to an adjustable motor configured to adjust resistance of pulling of the rope;
a display device disposed at a front surface of the fitness main body, wherein the display device comprises a mirror reflection layer to reflect an image of a user opposite the display device and electronic display layer to display video imagery of a fitness instructor;
a micro-camera and a 3D camera respectively disposed on top of the display device, wherein the micro-camera is configured to collect an optical image of the user, and the 3D camera is configured to obtain three-dimensional skeleton point data of the user, monitor, and adjust posture of the user, and
an intelligent control system operably coupled with at least one of the display device, micro-camera, 3D camera, and adjustable motor. The intelligent control system comprises a central controller to control at least one of the adjustable motor, display device, micro-camera, 3D camera, a biometric information unit, a video unit, a wireless communication module, a data collecting module, and a data processing module.

Preferably, the three-dimensional skeleton point data comprises multiple distances and intersection angels of different skeleton points of the user.

Preferably, the data collecting module is configured to collect data obtained by the 3D camera, and the data processing module is configured to process the data collected by the data collecting module by real-time person segmentation and 3D human pose estimation with fully convolutional model. The data processing module may also be configured to process the data collected by the data collecting module by semi-supervised approach and trajectory model. The data processing module is preferably configured to process the data collected by the data collecting module by real-time person segmentation to just capture the user's skeleton points and blur background, therefore protecting the user's privacy.

Preferably, the fitness device comprises a support arm substantially surrounding the rope.

Preferably, the fitness main body comprises a sliding rail at a side that is slidably connected with the support arm and the rope through a gear groove on the support arm.

More preferably, the fitness device comprises two of the ropes disposed at two sides of the fitness main body.

Preferably, the rope is retractable to at least one side of the fitness main body.

Preferably, the fitness device further comprises a wall mounting bracket at a back surface of the fitness main body for mounting the device to a wall.

Preferably, the display device is configured to display data output information comprising at least one of an optical image, an infrared image, a body fat rate, an exercise time, an exercise intensity, exercise effect analysis, and training morphology of the exerciser when turned on for exercise, and the display device is a mirror or is configured to display a dynamic or stationary painting in energy saving mode when turned off for exercise.

Preferably, a heart rate monitoring module is disposed on the handle.

Preferably, the central controller is configured to control a pull resistance force applied by the adjustable motor to the rope, and the electronic display layer is configured to display adjustment to the resistance force.

Preferably, the display device comprises a touch layer attached to the mirror reflection layer, and the central controller is configured to control the pull resistance force based on instruction received from the user via verbal command or the touch layer.

Preferably, the central controller is operably coupled to a backstage server configured to receive data of the user, store, analyze the data, or transmit analysis or video imagery to the user via the displaying device.

Preferably, a speaker and a microphone for audio transmission and online self-learning are disposed on the fitness main body.

Preferably, the mirror reflection layer has a height from about 40 inches to about 96 inches, preferably 55 inches to 90 inches, and a width from about 9 inches to about 120 inches, preferably 18 to 62 inches so that the mirror reflection layer is large enough to reflect a user in his or her real size (height and width).

Preferably, the intelligent control system is configured to allow the user to invite another user or other users to attend exercise together on another device or other devices from a different location or different locations and communicate with each other.

As an embodiment, the multifunctional and physiotherapy fitness device may include a fitness device body, a support arm, and an intelligent control system. A display device is disposed at a front surface of the fitness device body. The display device may be a high-reflection translucent coated glass, the high-reflection translucent coated glass may include a mirror reflection layer and a LED screen layer, a touch layer may be integrated to the mirror reflection layer, a display screen may be disposed on the touch layer, a camera device may be disposed on the top of the display device, the camera device may include a micro-camera and a 3D camera. The micro-camera may be configured to collect an optical image of an exerciser for communication between instructors and exercisers. The 3D camera may be configured to collect three-dimensional of skeleton point data to monitor and adjust posture of exercisers, monitor muscle exercise effect. In order to train deep learning neutral network model and loss function fast converge in artificial intelligence application, we extract 25 skeleton points and 15 combination of their distance as features. Meanwhile, we combine their intersection angles as features as well. In order to protect customer privacy and assist customer correct their posture instantly, we apply the real-time person segmentation and 3D human pose estimation with fully convolutional model and semi-supervised training method and edge computing technology. For the real-time person segmentation technology. Fully Convolutional networks comprise three-dimension array which is h*w*d. H and w represent spatial dimensions, and d represents feature dimension. When camera take the videos, the continue-image project to convolutional networks. All of layers of each image are convoluted. the final output layer will be the same height, width as the input images. Apply SoftMax function, the most likely class for each pixel of images will be found. The we can highlight the people's skeleton points but blur the people's background for the privacy purpose. Our approach of making computational complexity independent of key point spatial resolution can reach very high accuracy. The Mask R-CNN and cascaded pyramid network detections are more robust for 3D human pose estimation. The full convolution with residual connections, input as 2D, then transform them via temporal convolutions layers. The effectively controlling of the temporal receptive field is an effective way to implement 3D pose estimation. The concatenated (x,y) coordinates of the J points for each frame was handled by the first layer. The temporal convolution with kernel size W and C output channels was applied, which is followed by B ResNet-style blocks. The skip-connection encompasses it. Every block implements the 1D convolution with kernel size W and dilation factor D=WB, followed by a convolution with kernel size.

At this step, we normalize the batch, rectified units and dropout 10% feature to ensure model generalized. The receptive field exponentially increase when feature convolute to next layer, but the parameters accumulate linearly. The hyperparameters, W and D, are fixed. The last layer outputs a 3D pose prediction for all frames from the input sequence with temporal information. See FIG. 1, which shows an instantiation of our fully convolutional 3D pose estimation architecture. The input consists of 2D key points for a receptive field of 243 frames (B=4 blocks) with J=17 joints. Convolutional layers are in green where 2J, 3d1, 1024 denotes 2 J input channels, kernels of size 3 with dilation 1, and 1024 output channels. We also show tensor sizes in parentheses for a sample 1-frame prediction, where (243, 34) denotes 243 frames and 34 channels. Due to valid convolutions, we slice the residuals (left and right, symmetrically) to match the shape of subsequent tensors.

The semi-supervised approach and trajectory model are also applied to boost the accuracy. Labeled component and unlabeled one are combined to optimize. The ground truth 3D poses to handle the labeled data with supervised model. The encoder model is used to handle unlabeled data. We regress the 3D trajectory of the posture in order to optimize the back-projection to 2D. Each sample's weight uses the inverse of the ground truth depth. See FIG. 2 showing semi-supervised training with a 3D pose model which predict 2D poses as input. The regress the 3D trajectory of the person and add a self-constraint to match the mean bone lengths of the unlabeled predictions to be labeled one.

In computer vision, image segmentation refers to the technique of grouping pixels into semantic area to locate object and boundaries. We apply real time person segmentation to protect user's privacy which just captures users' skeleton points and blur the background. This method boosts accuracy of analysis as well. See FIG. 3. The algorithm calculates each single pixel by the clusters which sorted in order of the likelihood they model the background. Input pixels are matched against the corresponding cluster and classified according to whether the matching cluster is considered part of the background.

Algorithm: we model each pixel by a group of K clusters where each cluster consists of a weight wₖ and an average pixel value called the centroid cₖ.

The first step: In order to locate the match cluster in the group with the highest weight, we compare each of their pixels against the corresponding cluster group in segmenting input frames by decreasing sorted weights. To eliminating complexity of the algorithm, the Manhattan distance between the central and the input pixel is measured to evaluate the matching cluster because it use additions and subtraction.

The second step is adaptation. The initial pixel weight represents the probability of an input cluster differentiate into background. We set the rapidly change backgrounds as the higher initial weight and lower one in stationary background. When the matching cluster was defined, the overall clusters weights will be updated by:

∂*ₙ={∂ₙ₊1/β(1−∂ₙ), ₙ=h_{γ}

∂*ₙ={∂ₙ₊1/β(0−∂ₙ), ₙ=h_{γ}

where h_{γ} is the index of clusters for matching cluster. The parameter is the inverse of the learning rate.

The third step is regularization. The cluster weight represents how many times it has been matched. The high weights refer that the pixel has same color to the centroid. The cluster is more likely to model the background. The low weight refers the centroid color has not appeared very often, and the cluster classifies as the foreground object. We can set up the threshold of probability to classify it. We do regularization by below equation.

The step four is classification. The input pixels are classified by overall clusters weights those are weighted higher than the matched one. We apply the blow calculation after sorting:

Z=Σ_{n>hγ}ⁿ⁻¹∂

wherein Z is the proportion of the background accounted for by the high weighted clusters.

The support arms are disposed at both sides of the fitness device body through sliding rails respectively, a gear groove is disposed on the support arm, a gear fixing device for use in cooperation with the gear groove is disposed on the sliding rail, a rope is disposed inside the support arm, one end of the rope is penetrated through the support arm to connect with a handle and the other end of the rope is connected with an intelligent motor. The electromagnet switches are used to adjust the length, height, angles of two arms.

The intelligent control system includes a central controller, a posture recognition and analysis unit, a video unit, a wireless communication module, a data collecting module and a data processing module.

Preferably, a wall fixing device for installation is disposed at a back surface of the fitness device body.

Preferably, the support arms move freely on the sliding rail and is flexibly adjustable for position according to continuity requirements.

Preferably, the display device is configured to display data output information including an optical image, an 3D image, a body fat rate, a skin surface temperature, an exercise time, an exercise intensity, and exercise effect analysis of the exerciser; and the display device is an ordinary mirror when turned off. Also, it can display as a dynamic or stationary painting in low power consumption model.

Preferably, a heart rate monitoring module is disposed on the handle.

Preferably, a player and a microphone for audio transmission and online learning are disposed at a side surface of the fitness device body.

Preferably, the central controller controls a pull resistance applied by the intelligent motor to the rope to further control a size of an exercise force, and the display screen control and adjust the resistance scales. Meanwhile, the user's verbal commands can control the resistance scales as well.

Preferably, the device is connected with the cloud server through internet that stores the recorded videos.

Embodiments of the present invention have the following beneficial effects:

1) Stepless adjustments to both exercise counterweight and the exercise handle height can be implemented. The monitoring of exercise actions and effect can be realized by the 3D camera, the visible light camera, and the intelligent control. The adjustment and use of the entire fitness device can be realized by the intelligent module. Further, the frame of the fitness device occupies a smaller area and can be directly fixed onto the wall.

2) After being turned on, the display device can be used as a display screen which is connected with the cloud sever for fitness exercises though internet. According to video images, a user may carry out training as well as online teaching and learning. Meanwhile, the display screen displays training result analysis of the user including optical image training and correction, a body fat rate, an exercise time, an exercise intensity and an exercise effect analysis of the user, the user may observe his/her training morphology in front of the mirror so as to perform self-correction.

3) After being turned off, the display device can be used as an ordinary mirror. The two arms will be retracted into the device's left and right sides.

4) Features of a good external appearance, a smaller occupation space and ease of use and thus can help users to complete fitness trainings better.

### BRIEF DESCRIPTION OF THE DRAWINGS

FIG. 1 shows an instantiation of our fully convolutional 3D pose estimation architecture.

FIG. 2 shows semi-supervised training with a 3D pose model which predict 2D poses as input.

FIG. 3 shows the use of image segmentation to capture a users' skeleton points and blur the background.

FIG. 4 is a structural front view of an embodiment of the present invention.

FIG. 5 is a structural rear view of an embodiment of the present invention.

FIG. 6 is a structural side view of an embodiment of the present invention.

FIG. 7 is a diagram of use state of an embodiment of the present invention.

FIG. 8 is a block diagram of a system of an embodiment of the present invention.

FIG. 9 is skeleton and angles points designed for AI module based on an embodiment of the present invention.

### DETAILED DESCRIPTION OF PREFERRED EMBODIMENTS

To make the object, technical solutions and advantages of the present invention understood more clearly and easily, the technical solutions of the present invention will be clearly and fully described in combination with the accompanying drawings in the embodiments of the present invention. Apparently, the described embodiments are merely some embodiments of the present invention rather than all embodiments. Generally, the components of the embodiments of the present invention described in the drawings can be arranged and designed in different configurations.

In FIG. 9, the distance part is:

0: 4-9 Distance between hands and waist joints; 1: 7-12 Distance between hand and lumbar joints; 2: 2-4 Distance between hands and shoulders; 3: 5-7 Distance between hands and shoulders; 4: 0-4 Distance between head and hand; 5: 0-7 Distance between head and hands; 6: 4-10 Distance between knees and hands; 7: 7-13 Distance between knees and hands; 8: 4-17 The distance between the hands; 9: 11-14 The distance between the feet; 10: 10-13 The distance between the knees; 11: 6-10 Distance between elbows and knees; 12: 3-13 Distance between elbows and knees; 13: 4-23 Distance between hands and feet; 14: 7-20 Distance between hands and feet.

To extract more than the distance between the point because the change is most obvious in the fitness posture, such as the distance between two fingers, the distance between the two feet, head, respectively, and the left hand, right hand between the distance, in particular stance they have obvious change, but some distance is not necessary, such as 2 and 3 (shoulder and elbow) the distance between the two key points are adjacent to each other, no matter what is the cause of concrete action, as long as the positioning of the human body in front of the camera, it wouldn't change a set distance, they are the two adjacent nodes, thus avoided when designing the adjacent key point, And selected in the specific fitness 23 groups of posture differences between the most obvious 15 groups of distance characteristics stored in the designated file.

The Angle part is:

0: 2-3-4; 1: 5-6-7; 2: 9-10-11; 3: 12-13-14; 4: 3-2-1; 5: 6-5-1; 6: 10-8-13; 7: 7-12-13; 8: 4-9-10; 9: 4-0-7; 10: 4-8-7; 11: 1-8-13; 12: 1-8-10; 13: 4-1-8; 14: 7-1-8.

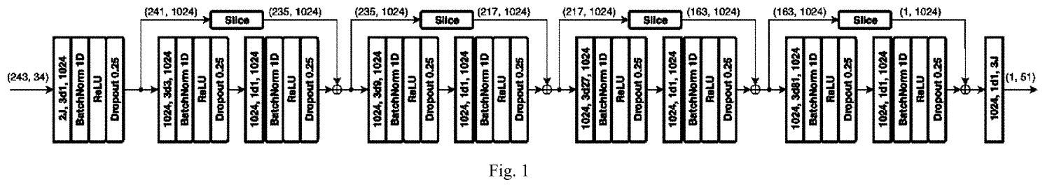

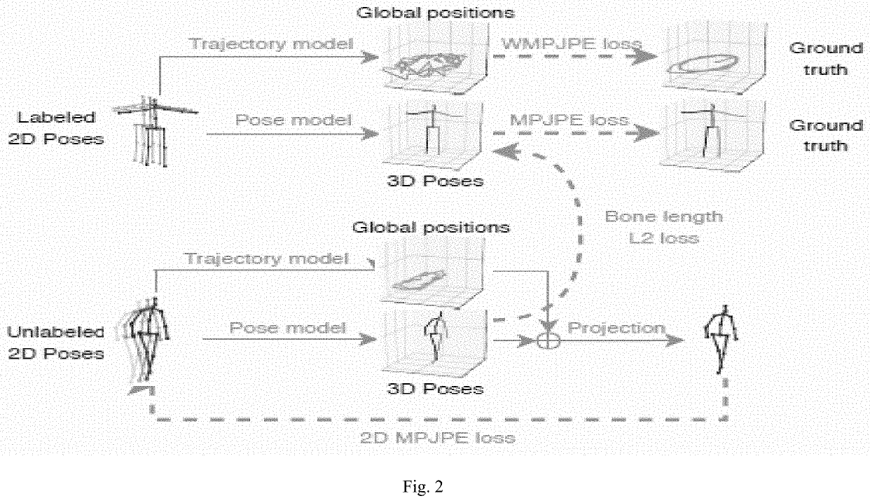

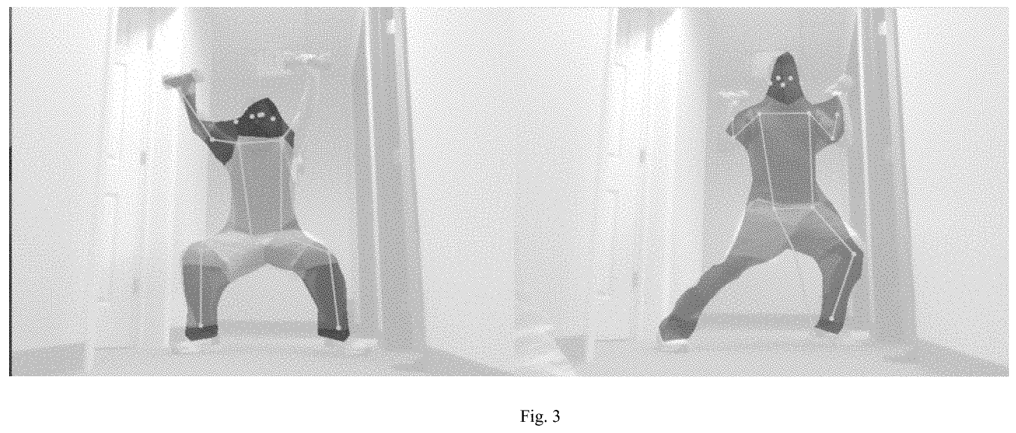

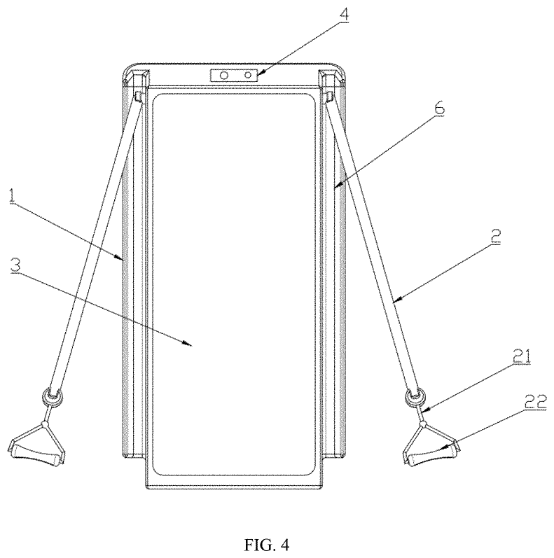

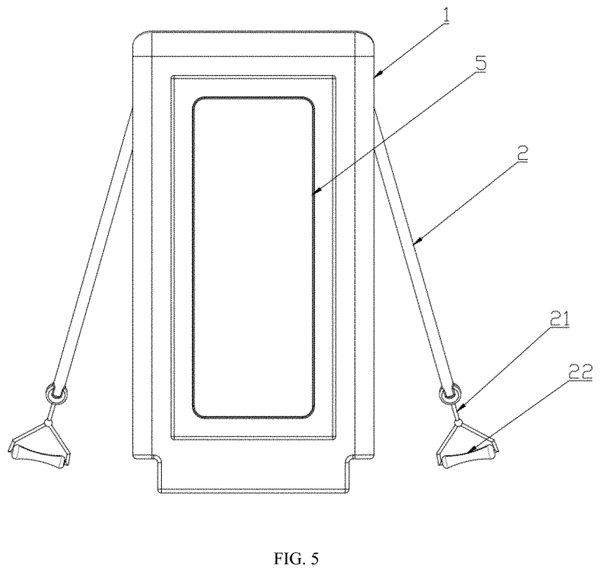

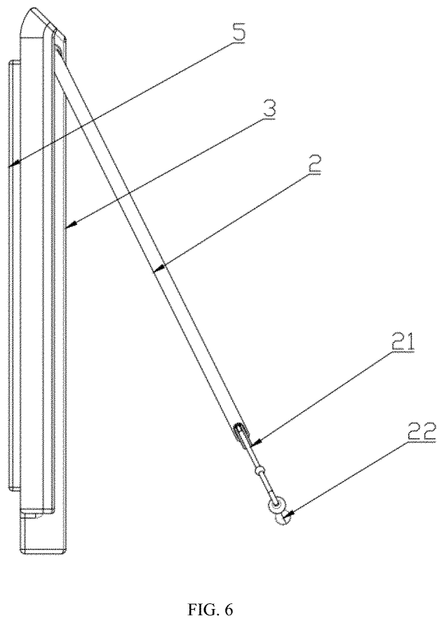

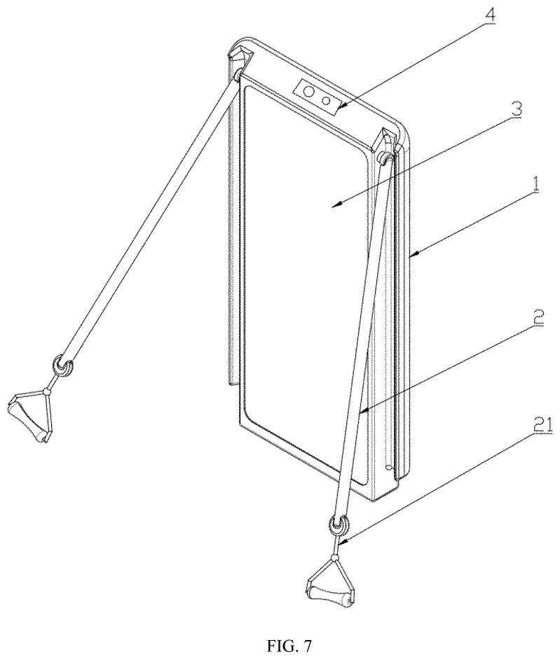

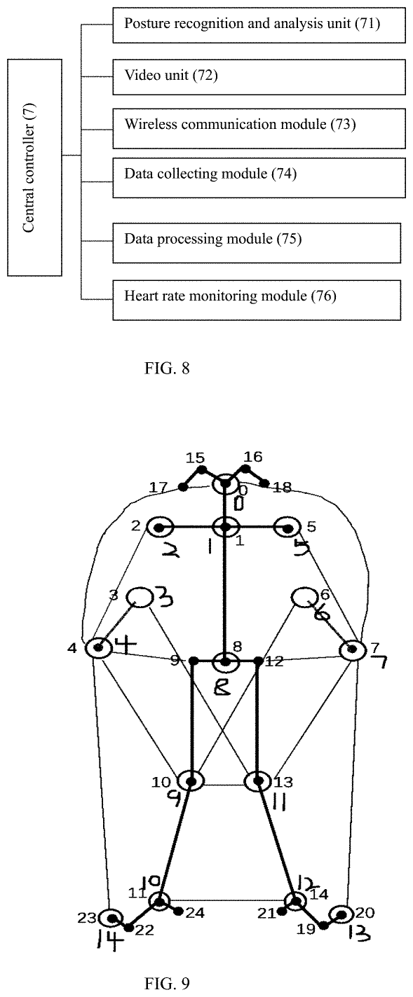

In FIGS. 1-9, numerals of drawings are described as follows:

1—Fitness device body, 2—support arm, 3—display device, 4—camera device, 5—wall fixing device, 6—sliding rail, 7—central controller, 71—Posture recognition and analysis unit, 72—video unit, 73—wireless communication module, 74—data collecting module, 75—data processing module, 76—heart rate monitoring module.

In the descriptions of the present invention, it is noted that orientations or positional relationship indicated by terms such as “central”, “upper”, “lower”, “left”, “right”, “vertical”, “horizontal”, “internal” and “external” are based on orientations or positional relationship indicated by the accompanying drawings. These orientations or positional relationships are used only to facilitate describing the present invention and simplifying the descriptions rather than indicate or imply that devices or elements indicated herein must have a particular orientation or are constructed and operated in a particular orientation and thus cannot be understood as limiting of the present invention.

As shown in figures, a multifunctional fitness device includes a fitness device body 1, a support arm 2 and an intelligent control system. A display device 3 is disposed at a front surface of the fitness device body 1. The display device 3 is a high-reflection translucent coated glass, the high-reflection translucent coated glass includes a mirror reflection layer and a LED display layer. A touch layer is attached to the mirror reflection layer, and a LED display is disposed on the touch layer; the display device 3 is configured to display data output information including an optical image, an multiple skeleton points image, a body fat rate, a skin surface temperature, an exercise time, an exercise intensity, and exercise effect analysis of an exerciser; and the display device 3 is an ordinary mirror when turned off.

A wall fixing device 5 for installation is disposed at a back surface of the fitness device body 1; a player and a microphone for audio transmission and online learning are disposed at a side surface of the fitness device body 1.

A camera device 4 is disposed at the top of the display device 3, the camera device 4 includes a micro-camera and a 3D camera. The micro-camera is configured to collect an optical image of the exerciser for oral communication between instructors and exercisers, and the 3D camera is configured to collect 25 skeleton points of the exerciser for action analysis and correction by AI.

The support arms 2 are disposed at both sides of the fitness device body 1 through sliding rails 6 respectively, a gear groove is disposed on the support arm 2, and a gear fixing device for use in cooperation with the gear groove is disposed on the sliding rail. The support arm 2 freely moves on the sliding rail and can be flexibly adjusted for position according to continuity requirements. A rope 21 is disposed inside the support arm 2, one end of the rope 21 is penetrated through the support arm 2 to connect with a handle 22 disposed with a heart rate monitoring module 76 and the other end of the rope 21 is connected with an intelligent motor.

The intelligent control system includes a central controller 7, a Posture recognition and analysis unit 71, a video unit 72, a wireless communication module 73, a data collecting module 74, and a data processing module 75. The central controller 7 controls a pull resistance applied by the intelligent motor to the rope 21 so as to further control a size of an exercise force. The display screen can realize adjustment to the size of the resistance. The central controller 7 sends collected data of user exercises to a backstage server which stores and analyzes the collected data. The backstage server is connected with a mobile terminal which is pre-installed with an application program adapted to a corresponding fitness appliance to realize intelligent control and intelligent analysis.

In use, training can be performed based on the application program preinstalled into the device. The application program read the videos contents stored on the cloud sever into the device. Grasping the support arms 2 with two hands and playing video images. The size of the exercise force can be wirelessly adjusted or adjusted by touching on the display screen or adjusted by user's oral command. The display screen will display optical image training and correction, a body fat rate, an exercise time, an exercise intensity, and an exercise effect analysis of the user and so on. Meanwhile, online teaching and online learning can be performed.

After being turned off, the display device 3 can be used as an ordinary mirror.

In the present invention, stepless adjustments to both exercise counterweight and the exercise handle height can be implemented, monitoring of exercise actions and exercise effect can be achieved by the 3D camera and visible light camera, and intelligent control, adjustment and use of the entire fitness device can be achieved by the intelligent module. Further the frame of the fitness device occupies a smaller area and can be directly fixed onto wall.

The present invention features a good external appearance, a smaller occupation space and ease of use and thus can help the user to complete fitness trainings better.

The foregoing is merely preferred embodiments of the present invention and shall not be intended to limit the present invention. Any modifications, equivalent substitutions and improvements and the like made within the spirit and principle of the present invention shall all fall within the scope of protection of the present invention.

I claim:

1. A fitness device comprising:
a fitness main body;
at least one rope slidably attached to the fitness main body, wherein the rope has two ends, with one end being connected to a handle, and the other end being connected to an adjustable motor configured to adjust resistance of pulling of the rope;
a display device disposed at a front surface of the fitness main body, wherein the display device comprises a mirror reflection layer to reflect an image of a user opposite the display device and electronic display layer to display video imagery of a fitness instructor;
a micro-camera and a 3D camera respectively disposed on top of the display device, wherein the micro-camera is configured to collect an optical image of the user, and the 3D camera is configured to obtain three-dimensional skeleton point data of the user, monitor, and adjust posture of the user, and
an intelligent control system operably coupled with at least one of the display device, micro-camera, 3D camera, and adjustable motor, wherein the intelligent control system comprises a central controller to control at least one of the adjustable motor, display device, micro-camera, 3D camera, a biometric information unit, a video unit, a wireless communication module, a data collecting module, and a data processing module.

2. The fitness device of claim 1 wherein the three-dimensional skeleton point data comprises multiple distances and intersection angels of different skeleton points of the user.

3. The fitness device of claim 1 wherein the data collecting module is configured to collect data obtained by the 3D camera, and the data processing module is configured to process the data collected by the data collecting module by real-time person segmentation and 3D human pose estimation with fully convolutional model.

4. The fitness device of claim 3 wherein the data processing module is also configured to process the data collected by the data collecting module by semi-supervised approach and trajectory model.

5. The fitness device of claim 3 wherein the data processing module is configured to process the data collected by the data collecting module by the following algorithms:
∂*ₙ={∂ₙ₊1/β(1−∂ₙ), ₙ=h_{γ}
∂*ₙ={∂ₙ₊1/β(0−∂ₙ), ₙ=h_{γ}
where h_{γ} is the index of clusters for matching cluster, the parameter is the inverse of the learning rate,
Z=Σ_{n>hγ}ⁿ⁻¹∂
wherein Z is the proportion of the background accounted for by the high weighted clusters.

6. The fitness device of claim 3 wherein the data processing module is configured to process the data collected by the data collecting module by real-time person segmentation to just capture the user's skeleton points and blur background, therefore protecting the user's privacy.

7. The fitness device of claim 1 comprising a support arm substantially surrounding the rope.

8. The fitness device of claim 7 wherein the fitness main body comprises a sliding rail at a side that is slidably connected with the support arm and the rope through a gear groove on the support arm.

9. The fitness device of claim 1 comprising two of the ropes disposed at two sides of the fitness main body.

10. The fitness device of claim 1 wherein the rope is retractable to at least one side of the fitness main body.

11. The fitness device of claim 1 further comprising a wall mounting bracket at a back surface of the fitness main body for mounting the device to a wall.

12. The fitness device of claim 1 wherein the display device is configured to display data output information comprising at least one of an optical image, an infrared image, a body fat rate, an exercise time, an exercise intensity, exercise effect analysis, and training morphology of the exerciser when turned on for exercise, and the display device is a mirror or is configured to display a dynamic or stationary painting in energy saving mode when turned off for exercise.

13. The fitness device according to claim 1, wherein a heart rate monitoring module is disposed on the handle.

14. The fitness device according to claim 1, wherein the central controller is configured to control a pull resistance force applied by the adjustable motor to the rope, and the electronic display layer is configured to display adjustment to the resistance force.

15. The fitness device according to claim 1, wherein the display device comprises a touch layer attached to the mirror reflection layer, the central controller is configured to control the pull resistance force based on instruction received from the user via verbal command or the touch layer.

16. The fitness device according to claim 1, wherein the central controller is operably coupled to a backstage server configured to receive data of the user, stores, analyzes the data, or transmits analysis or video imagery to the user via the displaying device.

17. The fitness device according to claim 1, wherein a speaker and a microphone for audio transmission and online self-learning are disposed on the fitness main body.

18. The fitness device according to claim 1, wherein the mirror reflection layer has a height from about 40 inches to about 96 inches and a width from about 9 inches to about 120 inches.

19. The fitness device according to claim 1 wherein the intelligent control system is configured to allow the user to invite another user or other users to attend exercise together on another device or other devices from a different location or different locations and communicate with each other.
